SEQUENCE LISTING (35 sequences)

SEQ ID NO: 1 - DNA, 405 bp, Aspergillus nidulans
  CDS (1)..(405): basf-dewA
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Aspergillus nidulans
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Aspergillus nidulans
  CDS (1)..(471): basf-rodA
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Aspergillus nidulans
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, Unknown
  CDS (1)..(336): basf-HypA
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, Unknown
  basf-HypA
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, Unknown
  CDS (1)..(357): basf-HypB
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, Unknown
  basf-HypB
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Schyzophyllum commune
  CDS (1)..(408): basf-sc3
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, Schyzophyllum commune
  basf-sc3
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Artificial sequence
  CDS (1)..(483): basf1
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Unknown
  basf1
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Artificial Sequence
  CDS (1)..(465): basf2
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Artificial Sequence
  basf2
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Artificial sequence
  CDS (1)..(882): basf-yaad
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, Artificial sequence
  basf-yaad
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Artificial sequence
  CDS (1)..(591): basf-yaae
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Artificial sequence
  basf-yaae
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Artificial sequence
  CDS (1)..(1329): basf-yaad-Xa-dewA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Artificial sequence
  basf-yaad-Xa-dewA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Artificial sequence
  CDS (1)..(1395): basf-yaad-Xa-rodA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Artificial sequence
  basf-yaad-Xa-rodA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Artificial sequence
  CDS (1)..(1407): basf-yaad-Xa-BASF1-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Artificial sequence
  basf-yaad-Xa-BASF1-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 28 bp, Unknown
  Hal570 oligonucleotide
gcgcgcccatggctcaaacaggtactga

SEQ ID NO: 26 - DNA, 28 bp, Unknown
  Hal571 oligonucleotide
gcagatctccagccgcgttcttgcatac

SEQ ID NO: 27 - DNA, 30 bp, Unknown
  Hal572 oligonucleotide
ggccatgggattaacaataggtgtactagg

SEQ ID NO: 28 - DNA, 33 bp, Unknown
  Hal573 oligonucleotide
gcagatcttacaagtgccttttgcttatattcc

SEQ ID NO: 29 - DNA, 38 bp, Unknown
  KaM416 oligonucleotide
gcagcccatcagggatccctcagccttggtaccagcgc

SEQ ID NO: 30 - DNA, 50 bp, Unknown
  KaM417 oligonucleotide
cccgtagctagtggatccattgaaggccgcatgaagttctccgtctccgc

SEQ ID NO: 31 - DNA, 45 bp, Unknown
  KaM434 oligonucleotide
gctaagcggatccattgaaggccgcatgaagttctccattgctgc

SEQ ID NO: 32 - DNA, 30 bp, Unknown
  KaM435 oligonucleotide
ccaatggggatccgaggatggagccaaggg

SEQ ID NO: 33 - DNA, 38 bp, Unknown
  KaM418 oligonucleotide
ctgccattcaggggatcccatatggaggagggagacag

SEQ ID NO: 34 - DNA, 32 bp, Unknown
  KaM464 oligonucleotide
cgttaaggatccgaggatgttgatgggggtgc

SEQ ID NO: 35 - DNA, 35 bp, Unknown
  KaM465 oligonucleotide
gctaacagatctatgttcgcccgtctccccgtcgt

(19) United States
(12) Patent Application Publication
(10) Pub. No.: US 2009/0241413 A1
(43) Pub. Date: Oct. 1, 2009

(54) Use of Proteins as an Antifoaming Constituent in Fuels

(75) Inventors: Thomas Subkowski, Ladenburg (DE); Hans-Georg Lemaire, Limburgerhof (DE); Marvin Karos, Schwetzingen (DE); Susan Hammer, Ludwigshafen (DE); Jörn Karl, Ludwigshafen (DE); Dietmar Posselt, Heidelberg (DE)

Correspondence Address: CONNOLLY BOVE LODGE & HUTZ, LLP, P O BOX 2207, WILMINGTON, DE, 19899, US

(73) Assignee: Basf Aktiengsellschaft, Ludwigshafen (DE)

(21) Appl. No.: 12/083,404

(22) PCT Filed: Oct. 9, 2006
(86) PCT No.: PCT/EP2006/067169

§ 371 Date: Apr. 10, 2008

(30) Foreign Application Priority Data
Oct. 12, 2005 (DE) 102005048720.3

(51) Int. Cl. C10L 1/222; C07K 14/37

(52) U.S. Cl. 44/405; 530/300

(57) ABSTRACT

The present invention relates to the use of at least one hydrophobin or of a derivative thereof as a defoamer in additive compositions or fuels, to a process for defoaming fuels, to an additive and fuel composition comprising at least one hydrophobin or derivative thereof and at least one further fuel additive, and to a process for preparing at least one fuel composition.

The present invention relates to the use of at least one hydrophobin or of a derivative thereof as a defoamer in additive compositions or fuels, to a process for defoaming fuels, to additive and fuel compositions comprising at least one hydrophobin or derivative thereof and at least one further fuel additive, and also to a process for producing at least one fuel composition.

The hydrocarbon mixtures used as fuel, which may also include aromatics, gas oil and kerosene, have the unpleasant property of developing foam in conjunction with air when they are transferred into stock vessels such as storage tanks and fuel tanks of motor vehicles. This leads to retardation of the transfer operation and to unsatisfactory filling of the vessels. It is therefore customary to add defoamers to the diesel fuel. These defoamers should be active in minimum concentration and must not form any damaging residues in the course of combustion of the diesel fuel in the engine or adversely affect the combustion of the fuel. Correspondingly active defoamers are described in the patent literature.

For instance, antifoams and defoamers based on silicon are known. DE 103 13 853 A discloses, for example, organofunctionally modified polysiloxanes and their use for defoaming liquid fuel, especially diesel fuel.

GB-B 2 173 510 relates to a process for defoaming diesel fuel or jet fuel, in which an antifoam based on a silicon polyether copolymer is added to the fuel.

One disadvantage of known antifoams is the poor defoaming of moist diesel fuel. Moist diesel fuel is understood to mean a fuel which includes approx. 250 ppm of water. This water is either water of condensation which gets into the fuel in the storage tanks or is introduced into the fuel during transport in oil tankers, as a result of the incomplete emptying of the tank of water.

It is also known from U.S. Pat. No. 5,542,960 that phenol derivatives (more preferably eugenol) exhibit a relatively good defoaming capacity in moist diesel fuel.

The antifoams described and further antifoams known from the prior art for diesel fuels feature various disadvantages. For instance, the silicon content of typical polysiloxane-polyoxyalkylene copolymers is from 10 to 15% by weight or even from 20 to 25% by weight. Since compounds with such a high silicon content can lead to undesired silicon dioxide deposits in the engine in the course of combustion, there is a desire for defoamers for diesel fuels with reduced silicon fraction or at least improved foam prevention and foam elimination, in order to be able to reduce the use concentration of these additives.

A further disadvantage of the known antifoams is that their compatibility (miscibility) with the additive packages which are added to the raw diesel oil to improve its properties is often too low. Additive packages are understood to mean mixtures of different additives, for example agents for improving the combustion performance, agents for reducing smoke formation, agents for reducing the formation of harmful exhaust gases, inhibitors for reducing the corrosion in the engine and its parts, interface-active substances, lubricants and the like. Such additive packages are described, for example, in JP-05 132 682, GB-2 248 068 and in the journal Mineralöltechnik, 37(4), 20. The additives of the additive package are dissolved in an organic solvent to give a stock concentrate which is added to the raw diesel fuel. Antifoams with polar groups frequently cannot be incorporated uniformly into these additive packages or separate in the course of storage.

One possible approach is that of naturally occurring additives which have the desired properties. A suitable variety of substances is present, for example, in the case of proteins.

Proteins are macromolecules which are formed from amino acids. The length of these polypeptide chains ranges from below 50, for example 10, up to over 1000 amino acids.

For the mode of action of the proteins, their three-dimensional structure is particularly important. The protein structure can be described by the primary structure, the secondary structure, the tertiary structure and the quaternary structure. The primary structure refers to the sequence of the individual amino acids within the polypeptide chain. The three-dimensional arrangement of the amino acids of a protein is referred to as the secondary structure. The tertiary structure is a three-dimensional arrangement of the polypeptide chain superordinate the secondary structure. It is determined by the forces and bonds between the residues (i.e. the side chains) of the amino acids. If a plurality of molecules in a three-dimensional arrangement form a superordinate functional unit, this is referred to as quaternary structure.

A distinction is drawn between two main groups of proteins, the globular proteins whose tertiary or quaternary structure has an approximately spherical or pear-shaped appearance and which are usually readily soluble in water or salt solutions, and the fibrillar proteins which have a thread-like or fibrous structure are usually insoluble and belong to the support and framework substances.

Hydrophobins are small proteins of from about 100 to 150 amino acids and are characteristic of filamentous fungi, for example Schizophyllum commune. They generally have 8 cysteine units.

Hydrophobins have a marked affinity for interfaces and are therefore suitable for coating surfaces in order to alter the properties of the interfaces by forming amphipathic membranes. For example, Teflon can be coated by means of hydrophobins to obtain a hydrophilic surface.

Hydrophobins can be isolated from natural sources. Likewise known are preparation methods for hydrophobins and derivatives thereof. For example, DE 10 2005 007 480.4 discloses a preparation process for hydrophobins and derivatives thereof.

Owing to the exceptional properties of hydrophobins for the coating of surfaces, these proteins have a high potential for numerous industrial applications. The prior art proposes the use of hydrophobins for various applications.

WO 96/41882 proposes the use of hydrophobins as emulsifiers, thickeners, surface-active substances, for the hydrophilization of hydrophobic surfaces, for the improvement of the water resistance of hydrophilic substrates, for the preparation of oil-in-water emulsions or of water-in-oil emulsions. Also proposed are pharmaceutical applications such as the production of ointments or creams, and also cosmetic applications such as skin protection or the production of hair shampoos or hair rinses. WO 96/41882 additionally claims compositions, especially compositions for pharmaceutical applications, comprising hydrophobins.

EP-A 1 252 516 discloses the coating of windows, contact lenses, biosensors, medical devices, vessels for carrying out experiments or for storage, ships' hulls, solid particles or frames or chassis of passenger vehicles with a solution comprising hydrophobins at a temperature of from 30 to 80° C.

WO 03/53383 discloses the use of hydrophobin for treating keratin materials in cosmetic applications.

WO 03/10331 discloses that hydrophobins have surface-active properties. For instance, a hydrophobin-coated sensor is disclosed, for example a test electrode, to which further substances, for example electroactive substances, antibodies or enzymes, are bonded in a noncovalent manner.

WO 2004/000880 likewise discloses the coating of surfaces with hydrophobin or hydrophobin-like substances. It is also disclosed that oil-in-water or water-in-oil emulsions can also be stabilized by adding hydrophobins.

WO 01/74864, which relates to hydrophobin-like proteins, also discloses that they can be used to stabilize dispersions and emulsions.

EP 05 007 208.1 proposes the use of proteins, especially of hydrophobins or derivates thereof, as demulsifiers.

Proceeding from the prior art, it was an object of the present invention to provide defoamers which have good defoaming action and have a low Si content.

It was a further object of the present invention to provide defoamers which, in addition to good defoaming action, are inexpensive.

It was a further object of the present invention to provide defoamers which, in addition to good defoaming action, are inexpensive and environmentally compatible.

According to the invention, this object is achieved by the use of at least one hydrophobin or of a derivative thereof as a defoamer in additive compositions or fuels.

The use of hydrophobins or derivatives thereof has the advantage that they are also naturally occurring substances which are biodegradable and thus do not lead to pollution of the environment. Moreover, the degradation forms hardly any substances which lead to deposits in the engine area.

According to the invention, hydrophobins or derivatives thereof are used as defoamers, i.e. the foam formation of a fuel or of a fuel composition is reduced.

According to the invention, it is possible to add at least one hydrophobin or a derivative thereof alone to a fuel as a defoamer. However, it is equally possible to use at least one hydrophobin or derivative thereof in combination with at least one further compound which acts as a defoamer. It is equally possible to use different hydrophobins or derivatives thereof in combination.

In the context of the present invention, a hydrophobin or a derivative thereof is understood to mean a hydrophobin or a modified hydrophobin. The modified hydrophobin may, for example, be a hydrophobin fusion protein or a protein which has a polypeptide sequence which has at least 60%, for example at least 70%, in particular at least 80%, more preferably at least 90%, especially preferably at least 95% identity with the polypeptide sequence of a hydrophobin, and which also satisfies the biological properties of a hydrophobin to an extent of 50%, for example to an extent of 60%, in particular to an extent of 70%, more preferably to an extent of 80%, especially the property that the surface properties are altered by coating with these proteins such that the contact angle of a water droplet before and after the coating of a glass surface with the protein is increased by at least 20°, preferably by at least 25°, in particular by at least 30°.

It has been found that, surprisingly, hydrophobins or derivatives thereof deliver good results in the case of use as defoamers.

For the definition of hydrophobins, what is crucial is the structural specificity and not the sequence specificity of the hydrophobins. The amino acid sequence of the mature hydrophobins is very diverse, but they all have a highly characteristic pattern of 8 conserved cysteine residues. These residues form four intramolecular disulfide bridges.

The N terminus and C terminus are variable over a relatively wide range. It is possible here to add on fusion partner proteins having a length of from 10 to 500 amino acids by means of molecular biology techniques known to those skilled in the art.

Moreover, hydrophobins and derivatives thereof are also understood in the context of the present invention to mean proteins with a similar structure and functional equivalence.

In the context of the present invention, the term “hydrophobins” should be understood hereinafter to mean polypeptides of the general structural formula (I)

Xₙ—C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I)

where X may be any of the 20 naturally occurring amino acids (Phe, Leu, Ser, Tyr, Cys, Trp, Pro, His, Gln, Arg, Ile Met, Thr, Asn, Lys, Val, Ala, Asp, Glu, Gly). In the formula, X may be the same or different in each case. The indices beside X are each the number of amino acids, C is cysteine, alanine, serine, glycine, methionine or threonine, where at least four of the residues designated with C are cysteine, and the indices n and m are each independently natural numbers between 0 and 500, preferably between 15 and 300.

The polypeptides of the formula (I) are also characterized by the property that, at room temperature, after coating a glass surface, they bring about an increase in the contact angle of a water droplet of at least 20°, preferably at least 25° and more preferably 30°, compared in each case with the contact angle of an equally large water droplet with the uncoated glass surface.

The amino acids designated with C¹ to C⁸ are preferably cysteines; however, they may also be replaced by other amino acids with similar space-filling, preferably by alanine, serine, threonine, methionine or glycine. However, at least four, preferably at least 5, more preferably at least 6 and in particular at least 7 of positions C¹ to C⁸ should consist of cysteines. In the inventive proteins, cysteines may either be present in reduced form or form disulfide bridges with one another. Particular preference is given to the intramolecular formation of C—C bridges, especially that with at least one intramolecular disulfide bridge, preferably 2, more preferably 3 and most preferably 4 intramolecular disulfide bridges. In the case of the above-described exchange of cysteines for amino acids with similar space-filling, such C positions are advantageously exchanged in pairs which can form intramolecular disulfide bridges with one another.

If cysteines, serines, alanines, glycines, methionines or threonines are also used in the positions designated with X, the numbering of the individual C positions in the general formulae can change correspondingly.

Preference is given to using hydrophobins of the general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

to perform the present invention, where X, C and the indices beside X and C are each as defined above, the indices n and m are each numbers between 0 and 300, and the proteins additionally feature the above-illustrated change in contact angle, and at least 6 of the residues designated with C are cysteine. More preferably, all C residues are cysteine.

Particular preference is given to using hydrophobins of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

where X, C and the indices besides X are each as defined above, the indices n and m are each numbers between 0 and 200, and the proteins additionally feature the above-illustrated change in contact angle.

The Xₙ and Xₘ residues may be peptide sequences which naturally are also joined to a hydrophobin. However, one or both residues may also be peptide sequences which are naturally not joined to a hydrophobin. This is also understood to mean those Xₙ and/or Xₘ residues in which a peptide sequence which occurs naturally in a hydrophobin is lengthened by a peptide sequence which does not occur naturally in a hydrophobin.

If Xₙ and/or Xₘ are peptide sequences which are not naturally bonded into hydrophobins, such sequences are generally at least 20, preferably at least 35, more preferably at least 50 and most preferably at least 100 amino acids in length. Such a residue which is not joined naturally to a hydrophobin will also be referred to hereinafter as a fusion partner. This is intended to express that the proteins may consist of at least one hydrophobin moiety and a fusion partner moiety which do not occur together in this form in nature.

The fusion partner moiety may be selected from a multitude of proteins. It is also possible for a plurality of fusion partners to be joined to one hydrophobin moiety, for example on the amino terminus (Xₙ) and on the carboxyl terminus (Xₘ) of the hydrophobin moiety. However, it is also possible, for example, for two fusion partners to be joined to one position (Xₙ or Xₘ) of the inventive protein.

Particularly suitable fusion partners are proteins which naturally occur in microorganisms, especially in E. coli or Bacillus subtilis. Examples of such fusion partners are the sequences yaad (SEQ ID NO: 15 and 16), yaae (SEQ ID NO: 17 and 18), and thioredoxin. Also very suitable are fragments or derivatives of these sequences which comprise only some, preferably from 70 to 99%, more preferably from 80 to 98% of the sequences mentioned, or in which individual amino acids or nucleotides have been changed compared to the sequence mentioned, in which case the percentages are each based on the number of amino acids.

The proteins used in accordance with the invention as hydrophobins or derivatives thereof may also be modified in their polypeptide sequence, for example by glycosilization, acetylation or else by chemical crosslinking, for example with glutaraldehyde.

One property of the hydrophobins or derivatives thereof used in accordance with the invention is the change in surface properties when the surfaces are coated with the proteins. The change in the surface properties can be determined experimentally, for example, by measuring the contact angle of a water droplet before and after the coating of the surface with the protein and determining the difference of the two measurements.

The performance of contact angle measurements is known in principle to those skilled in the art. The measurements are based on room temperature and water droplets of 5 μl. The precise experimental conditions for an example of a suitable method for measuring the contact angle are given in the experimental section. Under the conditions mentioned there, the proteins used in accordance with the invention have the property of increasing the contact angle by at least 20°, preferably at least 25°, more preferably at least 30°, compared in each case with the contact angle of an equally large water drop with the uncoated glass surface.

In the hydrophobin moiety of the hydrophobins or derivatives thereof known to date, the positions of the polar and nonpolar amino acids are conserved, which is manifested in a characteristic hydrophobicity plot. Differences in the biophysical properties and the hydrophobicity led to the division of the hydrophobins known to date into two classes, I and II (Wessels et al. 1994, Ann. Rev. Phytopathol., 32, 413-437).

The assembled membranes composed of class I hydrophobins are highly insoluble (even toward 1% sodium dodecylsulfate (SDS) at elevated temperature) and can only be dissociated again by concentrated trifluoroacetic acid (TFA) or formic acid. In contrast, the assembled forms of class II hydrophobins are less stable. They can be dissolved again merely by 60% ethanol or 1% SDS (at room temperature).

A comparison of the amino acid sequences shows that the length of the region between cysteine C³ and C⁴ in class II hydrophobins is distinctly shorter than in class I hydrophobins. Class II hydrophobins also have more charged amino acids than class I.

Particularly preferred hydrophobins for performing the present invention are the hydrophobins of the dewA, rodA, hypA, hypB, sc3, basf1, basf2 type, which are characterized structurally in the sequence listing which follows. They may also only be parts or derivatives thereof. It is also possible for a plurality of hydrophobin moieties, preferably 2 or 3, of the same or different structure to be bonded to one another and be bonded to a corresponding suitable polypeptide sequence which is naturally not joined to a hydrophobin.

Particularly suitable in accordance with the invention are also the fusion proteins with the polypeptide sequences shown in SEQ ID NO: 20, 22, 24, and also the nucleic acid sequences encoding them, especially the sequences according to SEQ ID NO: 19, 21, 23. Particularly preferred embodiments are also proteins which derive from the polypeptide sequences shown in SEQ ID NO. 20, 22 or 24 by virtue of exchange, insertion or deletion of at least one, up to 10, preferably 5, more preferably 5% of all amino acids, and which still have the biological property of the starting proteins to an extent of at least 50%. In this context, biological property of the proteins refers to the change in the contact angle by at least 20° already described.

Suitable fusion partners are proteins which lead to the fusion protein thus generated being capable of coating surfaces and simultaneously resistant toward a detergent treatment. Examples of fusion partners are, for example, yaad and yaae in E. coli, and thioredoxin.

It has been found that the fusion proteins produced in this way are functionally already active, and the hydrophobins do not, as described in the literature, have to be dissociated and thus activated by trifluoroacetic acid or formic acid treatment. Solutions which comprise these fusion proteins or, after cleavage of the fusion protein, comprise only the hydrophobin are suitable directly for the coating of surfaces.

At C- or N-terminal fusion with an affinity tag (for example His₆, HA, calmodulin-BD, GST, MBD, chitin-BD, streptavidin-BD-Avi Tag, Flag-Tag, T7, etc.) is found to be favorable for rapid and efficient purification. Corresponding standard protocols can be obtained from the commercial suppliers of the affinity tags.

A cleavage site between the hydrophobin and the fusion partner or the fusion partners can be utilized to release the pure hydrophobin in underivatized form (for example by BrCN cleavage at methionin, factor Xa cleavage, enterokinase cleavage, thrombin cleavage, TEV cleavage, etc.).

It is also possible to generate fusion proteins in succession from one fusion partner, for example yaad or yaae, and a plurality of hydrophobins, even of different sequence, for example DewA-RodA or Sc3-DewA, Sc3-RodA. It is equally possible to use hydrophobin fragments (for example N- or C-terminal truncations) or mutein which have up to 70% homology. The optimal constructs are in each case selected in relation to the particular use, i.e. the fuel to be defoamed.

The polypeptides used in accordance with the invention or present in the inventive compositions can be prepared chemically by known methods of peptide synthesis, for example by Merrifield solid-phase synthesis.

Naturally occurring hydrophobins can be isolated from natural sources by means of suitable methods. Reference is made by way of example to Wösten et. al., Eur. J. Cell Bio. 63, 122-129 (1994) or WO 96/41882.

Fusion proteins can be prepared preferably by genetic engineering methods, in which one nucleic acid sequence, especially DNA sequence, encoding the fusion partner and one encoding the hydrophobin moiety are combined in such a way that the desired protein is generated in a host organism as a result of gene expression of the combined nucleic acid sequence. Such a preparation process is disclosed, for example, in DE 102005007480.4.

Suitable host organisms (production organisms) for the preparation method mentioned may be prokaryotes (including the Archaea) or eukaryotes, particularly bacteria including halobacteria and methanococcia, fungi, insect cells, plant cells and mammalian cells, more preferably Escherichia coli, Bacillus subtilis, Bacillus megaterium, Aspergillus oryzae, Aspergillus nidulans, Aspergillus niger, Pichia pastoris, Pseudomonas spec., lactobacilli, Hansenula polymorpha, Trichoderma reesei, SF9 (or related cells), among others.

In this method, expression constructs comprising a nucleic acid sequence which encodes a polypeptide used in accordance with the invention, under the genetic control of regulatory nucleic acid sequences, and also vectors comprising at least one of these expression constructs, are used.

Constructs which are used preferably comprise a promoter 5′ upstream of the particular coding sequence and a terminator sequence 3′ downstream, and also, if appropriate, further customary regulatory elements, each linked operatively to the coding sequence.

In the context of the present invention, an “operative linkage” is understood to mean the sequential arrangement of promoter, coding sequence, terminator and, if appropriate, further regulatory elements, such that each of the regulatory elements can fulfill its function as intended in the expression of the coding sequence.

Examples of operatively linkable sequences are targeting sequences, and also enhancers, polyadenylation signals and the like. Further regulatory elements comprise selectable markers, amplification signals, replication origins and the like. Suitable regulatory sequences are, for example, described in Goeddel, Gene Expression Technology: Methods in Enzymology 185, Academic Press, San Diego, Calif. (1990).

In addition to these regulation sequences, the natural regulation of these sequences may still be present upstream of the actual structural genes and, if appropriate, have been genetically modified so as to switch off the natural regulation and increase the expression of the genes.

A preferred nucleic acid construct also advantageously comprises one or more so-called “enhancer” sequences, joined functionally to the promoter, which enable increased expression of the nucleic acid sequence. Also at the 3′ end of the DNA sequences, it is possible for additional advantageous sequences to be inserted, such as further regulatory elements or terminators.

The nucleic acids may be present in the construct in one or more copies. It is also possible for further markers such as antibiotic resistances or genes which complement auxotrophies to be present in the construct, if appropriate for selection for the construct.

Advantageous regulation sequences for the preparation are present, for example, in promoters such as the cos, tac, trp, tet, trp-tet, lpp, lac, lpp-lac, laclq-T7, T5, T3, gal-, trc, ara, rhaP(rhaPBAD) SP6, lambda-PR or imlambda-P promoter, which advantageously find use in Gram-negative bacteria. Further advantageous regulation sequences are present, for example, in the Gram-positive promoters amy and SP02, and in the yeast or fungal promoters ADC1, MFalpha, AC, P-60, CYC1, GAPDH, TEF, rp28, ADH.

It is also possible to use synthetic promoters for the regulation.

For expression in a host organism, the nucleic acid construct is advantageously inserted into a vector, for example a plasmid or a phage which enables optimal expression of the genes in the host. Apart from plasmids and phages, vectors are also understood to mean all other vectors known to those skilled in the art, for example viruses such as SV40, CMV, baculovirus and adenovirus, transposons, IS elements, phasmids, cosmids, and linear or circular DNA, and also the Agrobacterium system.

These vectors can be replicated autonomously in the host organism or replicated chromosomally. Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, plN-III″3-B1, tgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alpha, pAG-1, YEp6, YEp13 or pEMBLYe23 or in plants pLGV23, pGHlac+pBIN19, pAK2004 or pDH51. The plasmids mentioned constitute a small selection of the possible plasmids. Further plasmids are known to those skilled in the art and can be taken, for example, from the book Cloning Vectors (Eds. Pouwels P. H. et al. Elsevier, Amsterdam-New York-Oxford, 1985, ISBN 0 444 904018).

Advantageously, the nucleic acid construct, for the expression of the further genes present, additionally also comprises 3′- and/or 5′-terminal regulatory sequences for enhancing the expression, which are selected for optimal expression depending upon the host organism and gene or genes selected.

These regulatory sequences are intended to enable the controlled expression of the genes and of the protein expression. Depending on the host organism, this can mean, for example, that the gene is expressed or overexpressed only after induction, or that it is expressed and/or overexpressed immediately.

The regulatory sequences or factors can preferably positively influence and thus increase the gene expression of the genes introduced. Thus, an amplification of the regulatory elements can advantageously be effected at the transcription level by using strong transcription signals such as promoters and/or enhancers. In addition, it is also possible to enhance the translation by, for example, improving the stability of the mRNA.

In a further embodiment of the vector, the vector comprising the nucleic acid construct or the nucleic acid can also be introduced into the microorganisms advantageously in the form of a linear DNA and be integrated into the genome of the host organism by means of heterologous or homologous recombination. This linear DNA can consist of a linearized vector such as a plasmid or only of the nucleic acid construct or the nucleic acid.

For an optimal expression of heterologous genes in organisms, it is advantageous to alter the nucleic acid sequences in accordance with the specific “codon usage” used in the organism. The “codon usage” can be determined easily with reference to computer evaluations of other, known genes of the organism in question.

An expression cassette is prepared by fusion of a suitable promoter with a suitable coding nucleotide sequence and a terminator signal or polyadenylation signal. To this end, common recombination and cloning techniques are used, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector which enables an optimal expression of the genes in the host. Vectors are well known to those skilled in the art and can be taken, for example, from “Cloning Vectors” (Pouwels P. H. et al., eds., Elsevier, Amsterdam-New York-Oxford, 1985).

With the aid of vectors, it is possible to prepare recombinant microorganisms which have been transformed, for example, with at least one vector and can be used for the production of the hydrophobins or derivatives thereof used in accordance with the invention. Advantageously, the above-described recombinant constructs are introduced into a suitable host system and expressed. Preference is given to using the cloning and transfection methods familiar to those skilled in the art, for example coprecipitation, protoplast fusion, electroporation, retroviral transfection and the like, in order to bring about the expression of the nucleic acids mentioned in the particular expression system. Suitable systems are described, for example, in Current Protocols in Molecular Biology, F. Ausubel et al., ed., Wiley Interscience, New York 1997, or Sambrook et al. Molecular Cloning: A Laboratory Manual, 2nd edition, Cold Spring Harbor Laboratory, Cold Spring Harbor Laboratory Press, Cold Spring Harbor, N.Y., 1989.

It is also possible to prepare homologously recombined microorganisms. To this end, a vector is prepared which comprises at least a section of a gene to be used or a coding sequence, in which, if appropriate, at least one amino acid deletion, additional or substitution has been introduced in order to change, for example to functionally disrupt, the sequence (“knockout” vector). The sequence introduced may, for example, also be a homolog from a related microorganism or be derived from a mammalian, yeast or insect source. The vector used for the homologous recombination may alternatively be configured such that the endogenous gene in the case of homologous recombination has been mutated or altered in another way, but still encodes the functional protein (for example, the upstream regulatory region can be changed such that the expression of the endogenous protein is changed). The changed section of the gene used in accordance with the invention is in the homologous recombination vector. The construction of suitable vectors for homologous recombination is described, for example, in Thomas, K. R. and Capecchi, M. R. (1987) Cell 51:503.

In principle, all prokaryotic or eukaryotic organisms are useful as recombinant host or ganisms for such nucleic acids or such nucleic acid constructs. Advantageously, the host organisms used are microorganisms such as bacteria, fungi or yeasts. Advantageously, Gram-positive or Gram-negative bacteria are used, preferably bacteria from the families Enterobacteriaceae, Pseudomonadaceae, Rhizobiaceae, Streptomycetaceae or Nocardiaceae, more preferably bacteria of the genera Escherichia, Pseudomonas, Streptomyces, Nocardia, Burkholderia, Salmonella, Agrobacterium or Rhodococcus.

The organisms used in the above-described preparation processes for fusion proteins are, depending on the host organism, grown or cultured in a manner known to those skilled in the art. Microorganisms are generally grown in a liquid medium which comprises a carbon source, usually in the form of sugars, a nitrogen source, usually in the form of organic nitrogen sources such as yeast extract or salts such as ammonium sulfate, trace elements such as iron, manganese and magnesium salts, and also, if appropriate, vitamins, at temperatures between 0 and 100° C., preferably between 10 to 60° C., with oxygen sparging. The pH of the nutrient liquid can be kept at a fixed value, i.e. is regulated or not during the growth. The growth can be effected batchwise, semi-batchwise or continuously. Nutrients can be introduced at the start of the fermentation or be replenished semicontinuously or continuously. The enzymes can be isolated from the organisms by the process described in the examples or be used for the reaction as a crude extract.

The proteins used in accordance with the invention, or functional, biologically active fragments thereof, can be prepared by means of a process for recombinant preparation, in which a polypeptide-producing microorganism is cultivated, the expression of the proteins is induced if appropriate and they are isolated from the culture. The proteins can also be produced in this way on an industrial scale if this is desired. The recombinant microorganism can be cultivated and fermented by known processes. Bacteria can be propagated, for example, in TB or LB medium and at a temperature of from 20 to 40° C. and a pH of from 6 to 9. Suitable cultivation conditions are described specifically in T. Maniatis, E. F. Fritsch and J. Sambrook, Molecular Cloning: A Laboratory Manual, Cold Spring Harbor Laboratory, Cold Spring Harbor, N.Y. (1989).

If the proteins are not secreted into the culture medium, the cells are then disrupted and the product is obtained from the lysate by known protein isolation processes. As desired, the cells can be disrupted by high-frequency ultrasound, by high pressure, for example in a French pressure cell, by osmolysis, by the action of detergents, lytic enzymes or organic solvents, by homogenizers or by combination of a plurality of the processes listed.

The proteins can be purified by known chromatographic processes, such as molecular sieve chromatography (gel filtration) such as Q Sepharose chromatography, ion exchange chromatography and hydrophobic chromatography, and also with other customary processes such as ultrafiltration, crystallization, salting-out, dialysis and native gel electrophoresis. Suitable processes are described, for example, in Cooper, F. G., Biochemische Arbeitsmethoden [Biochemical Techniques], Verlag Walter de Gruyter, Berlin, New York, or in Scopes, R., Protein Purification, Springer Verlag, New York, Heidelberg, Berlin.

It may be advantageous to isolate the recombinant protein by using vector systems or oligonucleotides which extend the cDNA by certain nucleotide sequences and hence encode altered polypeptides or fusion proteins which serve, for example, for simpler purification. Such suitable modifications comprise so-called “tags” which function as anchors, for example the modification known as the hexa-histidine anchor, or epitopes which can be recognized as antigens of antibodies (described, for example, in Harlow, E. and Lane, D., 1988, Antibodies: A Laboratory Manual, Cold Spring Harbor (N.Y.) Press). Further suitable tags are, for example, HA, calmodulin-BD, GST, MBD; Chitin-BD, streptavidin-BD-Avi Tag, Flag-Tag, T7 etc. These anchors may serve, for example, to attach the proteins to a solid support, for example a polymer matrix, which can be introduced, for example, into a chromatography column, or be used on a microtiter plate or on another support. The corresponding purification protocols are obtainable from the commercial affinity tag suppliers.

The proteins prepared as described may be used either directly as fusion proteins or, after detachment and removal of the fusion partner, as “pure” hydrophobins.

When a removal of the fusion partner is intended, it is advisable to incorporate a potential cleavage site (specific recognition site for proteases) into the fusion protein between hydrophobin moiety and fusion partner moiety. Suitable cleavage sites are especially those peptide sequences which otherwise occur neither in the hydrophobin moiety nor in the fusion partner moiety, which can be determined easily with bioinformatic tools. Particularly suitable are, for example, BrCN cleavage at methionine, or protease-mediated cleavage with factor Xa cleavage, enterokinase cleavage, thrombin cleavage or TEV (Tobacco etch virus Protease) cleavage.

In the context of the present invention, fuels are understood to mean both fuels in the narrower sense, which are used to operate internal combustion engines, and fuels in general.

Suitable fuels are middle distillates and gasoline fuels. However, preference is given to using middle distillates.

Suitable middle distillates are those which boil in a range of from 120 to 500° C. and are selected, for example, from diesel fuels, kerosene and heating oil. Preferred middle distillates are diesel fuels.

The diesel fuels are, for example, crude oil raffinates which typically have a boiling range of from 100 to 400° C. These are usually distillates having a 95% point up to 360° C. or even higher. However, they may also be “ultra-low sulfur diesel” or “city diesel”, characterized by a 95% point of, for example, not more than 345° C. and a sulfur content of not more than 0.005% by weight, or by a 95% point of, for example, 285° C. and a sulfur content of not more than 0.001% by weight. In addition to the diesel fuels obtainable by refining, those which are obtainable by coal gasification or gas liquefaction (“gas-to-liquid” (GTL) fuels) are suitable. Also suitable are mixtures of the aforementioned diesel fuels with renewable fuels such as biodiesel or bioethanol.

The diesel fuels are more preferably those having a low sulfur content, i.e. having a sulfur content of less than 0.05% by weight, preferably of less than 0.02% by weight, in particular of less than 0.005% by weight and especially of less than 0.001% by weight of sulfur. The heating oils are also more preferably those having a low sulfur content, for example having a sulfur content of at most 0.1% by weight, preferably of at most 0.05% by weight, more preferably of at most 0.005% by weight and in particular of at most 0.001% by weight.

Preference is given in accordance with the invention to using hydrophobins or derivatives thereof as defoamers in diesel fuels.

In a further embodiment, the present invention therefore relates to use as described above of at least one hydrophobin or of a derivative thereof as a defoamer, wherein the fuel is a diesel fuel.

According to the invention, the at least one hydrophobin or derivative thereof is used preferably in an amount of from 0.01 to 100 ppm based on the fuel, preferably of from 0.15 to 50 ppm, more preferably of from 0.2 to 30 ppm or from 0.3 to 10 ppm.

In the context of the present application, the unit ppm means mg per kg.

In a further embodiment, the present invention therefore relates to use as described above of at least one hydrophobin or of a derivative thereof as a defoamer, wherein the at least one hydrophobin or derivative thereof is used in an amount of from 0.1 to 100 ppm based on the fuel.

According to the invention, a fuel, especially a diesel fuel, can be defoamed by adding at least one hydrophobin or a derivative thereof.

The present invention therefore also relates to a process for defoaming fuel, comprising the addition of at least one hydrophobin or derivative thereof to a fuel.

In a further embodiment, the present invention relates to a process as described above for defoaming fuel, wherein the fuel is a diesel fuel.

In a further preferred embodiment, the present invention relates to a process as described above for defoaming fuel, wherein the at least one hydrophobin or derivative thereof is used in an amount of from 0.1 to 100 ppm based on the fuel.

It is possible in the context of the present invention that the at least one hydrophobin or derivative thereof is added directly to a fuel or to a fuel composition or in the form of an additive composition.

The present invention further relates to additive compositions which, in addition to at least one further fuel additive, comprise at least one hydrophobin or a derivative thereof. The present invention likewise relates to fuel compositions which comprise at least one hydrophobin or a derivative thereof and at least one further fuel additive.

In a further embodiment, the present invention therefore relates to an additive composition comprising at least one hydrophobin or derivative thereof and at least one further fuel additive.

In a further embodiment, the present invention likewise relates to a fuel composition comprising, in addition to at least one fuel as a main constituent, at least one hydrophobin or derivative thereof and at least one further fuel additive.

The additive composition or the fuel comprise, in addition to the at least one hydrophobin or derivative thereof, at least one further fuel additive, especially at least one detergent and/or a demulsifier. Suitable detergent additives and demulsifiers are listed below. The additive compositions and fuels may also comprise, instead or in addition, various fuel additives such as carrier oils, corrosion inhibitors, antioxidants, antistats, dye markers and the like. However, the additive composition or the fuel preferably comprise at least one detergent and/or a demulsifier and, if appropriate, further different fuel additives.

In a further preferred embodiment, the present invention therefore relates to an additive composition or fuel composition as described above, wherein the composition comprises at least one detergent. In a further preferred embodiment, the present invention likewise relates to an additive composition or fuel composition as described above, wherein the composition comprises at least one demulsifier.

Suitable detergent additives are listed by way of example hereinafter.

The detergent additives are preferably amphiphilic substances which have at least one hydrophobic hydrocarbyl radical having a number-average molecular weight (Mn) of from 85 to 20 000 and at least one polar moiety selected from:
(a) mono- or polyamino groups having up to 6 nitrogen atoms, of which at least one nitrogen atom has basic properties;
(b) nitro groups, if appropriate in combination with hydroxyl groups;
(c) hydroxyl groups in combination with mono- or polyamino groups, in which at least one nitrogen atom has basic properties;
(d) carboxyl groups or their alkali metal or their alkaline earth metal salts;
(e) sulfonic acid groups or their alkali metal or alkaline earth metal salts;
(f) polyoxy-C₂- to -C₄-alkylene groups which are terminated by hydroxyl groups, mono- or polyamino groups, in which at least one nitrogen atom has basic properties, or by carbamate groups;
(g) carboxylic ester groups;
(h) moieties derived from succinic anhydride and having hydroxyl and/or amino and/or amido and/or imido groups; and/or
(i) moieties obtained by Mannich reaction of substituted phenols with aldehydes and mono- or polyamines.

The hydrophobic hydrocarbyl radical in the above detergent additives, which ensures the adequate solubility in the fuel, has a number-average molecular weight (Mn) of from 85 to 20 000, especially from 113 to 10 000, in particular from 300 to 5000. Typical hydrophobic hydrocarbyl radicals, especially in conjunction with the polar moieties (a), (c), (h) and (i), include polypropenyl, polybutenyl and polyisobutenyl radicals each having Mn=from 300 to 5000, especially from 500 to 2500, in particular from 700 to 2300.

Examples of the above groups of detergent additives include the following:

Additives comprising mono- or polyamino groups (a) are preferably polyalkenemono- or polyalkenepolyamines based on polypropene or conventional (i.e. having predominantly internal double bonds) polybutene or polyisobutene having Mn=from 300 to 5000. When polybutene or polyisobutene having predominantly internal double bonds (usually in the beta and gamma position) are used as starting materials in the preparation of the additives, a possible preparative route is by chlorination and subsequent amination or by oxidation of the double bond with air or ozone to give the carbonyl or carboxyl compound and subsequent amination under reductive (hydrogenating) conditions. The amines used here for the amination may be, for example, ammonia, monoamines or polyamines, such as dimethylaminopropylamine, ethylenediamine, diethylenetriamine, triethylenetetramine or tetraethylenepentamine. Corresponding additives based on polypropene are described in particular in WO 94/24231.

Further preferred additives comprising monoamino groups (a) are the hydrogenation products of the reaction products of polyisobutenes having an average degree of polymerization P of from 5 to 100 with nitrogen oxides or mixtures of nitrogen oxides and oxygen, as described in particular in WO 97/03946.

Further preferred additives comprising monoamino groups (a) are the compounds obtainable from polyisobutene epoxides by reaction with amines and subsequent dehydration and reduction of the amino alcohols, as described in particular in DE-A 196 20 262.

Additives comprising nitro groups (b), if appropriate in combination with hydroxyl groups, are preferably reaction products of polyisobutenes having an average degree of polymerization P of from 5 to 100 or from 10 to 100 with nitrogen oxides or mixtures of nitrogen oxides and oxygen, as described in particular in WO 96/03367 and WO 96/03479. These reaction products are generally mixtures of pure nitropolyisobutenes (e.g. α,β-dinitropolyisobutene) and mixed hydroxynitropolyisobutenes (e.g. α-nitro-β-hydroxypolyisobutene).

Additives comprising hydroxyl groups in combination with mono- or polyamino groups (c) are in particular reaction products of polyisobutene epoxides obtainable from polyisobutene having preferably predominantly terminal double bonds and Mn from 300 to 5000, with ammonia or mono- or polyamines, as described in particular in EP-A 476 485.

Additives comprising carboxyl groups or their alkali metal or alkaline earth metal salts (d) are preferably copolymers of C₂-C₄₀-olefins with maleic anhydride which have a total molar mass of from 500 to 20 000 and of whose carboxyl groups some or all have been converted to the alkali metal or alkaline earth metal salts and any remainder of the carboxyl groups has been reacted with alcohols or amines. Such additives are disclosed in particular by EP-A 307 815. Such additives serve mainly to prevent valve seat wear and can, as described in WO 87/01126, advantageously be used in combination with customary fuel detergents such as poly(iso)buteneamines or polyetheramines.

Additives comprising sulfonic acid groups or their alkali metal or alkaline earth metal salts (e) are preferably alkali metal or alkaline earth metal salts of an alkyl sulfosuccinate, as described in particular in EP-A 639 632. Such additives serve mainly to prevent valve seat wear and can be used advantageously in combination with customary fuel detergents such as poly(iso)buteneamines or polyetheramines.

Additives comprising polyoxy-C₂-C₄-alkylene moieties (f) are preferably polyethers or polyetheramines which are obtainable by reaction of C₂- to C₆₀-alkanols, C₆- to C₃₀-alkanediols, mono- or di-C₂-C₃₀-alkylamines, C₁-C₃₀-alkylcyclohexanols or C₁-C₃₀-alkylphenois with from 1 to 30 mol of ethylene oxide and/or propylene oxide and/or butylene oxide per hydroxyl group or amino group and, in the case of the polyetheramines, by subsequent reductive amination with ammonia, monoamines or polyamines. Such products are described in particular in EP-A 310 875, EP-A 356 725, EP-A 700 985 and U.S. Pat. No. 4,877,416. In the case of polyethers, such products also have carrier oil properties. Typical examples of these are tridecanol butoxylates, isotridecanol butoxylates, isononylphenol butoxylates and polyisobutenol butoxylates and propoxylates and also the corresponding reaction products with ammonia.

Additives comprising carboxylic ester groups (g) are preferably esters of mono-, di- or tricarboxylic acids with long-chain alkanols or polyols, in particular those having a minimum viscosity of 2 mm²/s at 100° C., as described in particular in DE-A 38 38 918. The mono-, di- or tricarboxylic acids used may be aliphatic or aromatic acids, and particularly suitable ester alcohols or ester polyols are long-chain representatives having, for example, from 6 to 24 carbon atoms. Typical representatives of the esters are adipates, phthalates, isophthalates, terephthalates and trimellitates of isooctanol, of isononanol, of isodecanol and of isotridecanol. Such products also have carrier oil properties.

Additives comprising moieties derived from succinic anhydride and having hydroxyl and/or amino and/or amido and/or imido groups (h) are preferably corresponding derivatives of polyisobutenylsuccinic anhydride which are obtainable by reacting conventional or highly reactive polyisobutene having Mn=from 300 to 5000 with maleic anhydride by a thermal route or via the chlorinated polyisobutene. Particular interest attaches to derivatives with aliphatic polyamines such as ethylenediamine, diethylenetriamine, triethylenetetramine or tetraethylenepentamine. The moieties having hydroxyl and/or amino and/or amido and/or imido groups are, for example, carboxylic acid groups, acid amides, acid amides of di- or polyamines which, in addition to the amide function, also have free amine groups, succinic acid derivatives having an acid and an amide function, carboximides with monoamines, carboximides with di- or polyamines which, in addition to the imide function, also have free amine groups, and diimides which are formed by the reaction of di- or polyamines with two succinic acid derivatives. Such fuel additives are described in particular in U.S. Pat. No. 4,849,572.

Additives (i) comprising moieties obtained by Mannich reaction of substituted phenols with aldehydes and mono- or polyamines are preferably reaction products of polyisobutene-substituted phenols with formaldehyde and mono- or polyamines such as ethylenediamine, diethylenetriamine, triethylenetetramine, tetraethylenepentamine or dimethylaminopropylamine. The polyisobutenyl-substituted phenols may stem from conventional or highly reactive polyisobutene having Mn=from 300 to 5000. Such “polyisobutene-Mannich bases” are described in particular in EP-A 831 141.

For a more precise definition of the fuel additives detailed individually, reference is explicitly made here to the disclosures of the abovementioned prior art documents.

Particular preference is given to detergent additives from group (h). These are in particular polyisobutenyl-substituted succinimides, especially the imides with aliphatic polyamines.

Examples of demulsifiers suitable in accordance with the invention include the following.

Demulsifiers are substances which bring about the demixing of an emulsion. They may be either ionogenic or nonionogenic substances which are effective at the phase boundary. Accordingly, all surface-active substances are in principle suitable as demulsifiers. Particularly suitable demulsifiers are selected from anion-active compounds such as the alkali metal or alkaline earth metal salts of alkyl-substituted phenol- and naphthalenesulfonates and the alkali metal or alkaline earth metal salts of fatty acids, and also uncharged compounds such as alcohol alkoxylates, e.g. alcohol ethoxylates, phenol alkoxylates, e.g. tert-butylphenol ethoxylate or tert-pentylphenol ethoxylate, fatty acids, alkylphenols, condensation products of ethylene oxide (EO) and propylene oxide (PO), for example also in the form of EO/PO block copolymers, polyethyleneimines or else polysiloxanes.

The additive composition and the fuel may additionally be combined with further customary components and additives. Mention should be made here, for example, of carrier oils without marked detergent action, these being employed in particular in the case of use in gasoline fuels. However, they are occasionally also used in middle distillates.

Suitable carrier oils are listed by way of example hereinbelow.

Suitable mineral carrier oils are the fractions obtained in crude oil processing, such as brightstock or base oils having viscosities, for example, from the SN 500-2000 class; and also aromatic hydrocarbons, paraffinic hydrocarbons and alkoxyalkanols. Likewise useful is a fraction which is obtained in the refining of mineral oil and is known as “hydrocrack oil” (vacuum distillate cut having a boiling range of from about 360 to 500° C., obtainable from natural mineral oil which has been catalytically hydrogenated under high pressure and isomerized and also deparaffinized). Likewise suitable are mixtures of abovementioned mineral carrier oils.

Examples of synthetic carrier oils which are useful in accordance with the invention are selected from: polyolefins (poly-alpha-olefins or poly(internal olefin)s), (poly)esters, (poly)alkoxylates, polyethers, aliphatic polyetheramines, alkylphenol-started polyethers, alkylphenol-started polyetheramines and carboxylic esters of long-chain alkanols.

Examples of suitable polyolefins are olefin polymers having Mn=from 400 to 1800, in particular based on polybutene or polyisobutene (hydrogenated or nonhydrogenated).

Examples of suitable polyethers or polyetheramines are preferably compounds comprising polyoxy-C₂-C₄-alkylene moieties which are obtainable by reacting C₂-C₆₀-alkanols, C₆-C₃₀-alkanediols, mono- or di-C₂-C₃₀-alkylamines, C₁-C₃₀-alkylcyclo-hexanols or C₁-C₃₀-alkylphenols with from 1 to 30 mol of ethylene oxide and/or propylene oxide and/or butylene oxide per hydroxyl group or amino group, and, in the case of the polyetheramines, by subsequent reductive amination with ammonia, monoamines or polyamines. Such products are described in particular in EP-A 310 875, EP-A 356 725, EP-A 700 985 and U.S. Pat. No. 4,877,416. For example, the polyetheramines used may be poly-C₂-C₆-alkylene oxide amines or functional derivatives thereof. Typical examples thereof are tridecanol butoxylates or isotridecanol butoxylates, isononylphenol butoxylates and also polyisobutenol butoxylates and propoxylates, and also the corresponding reaction products with ammonia.

Examples of carboxylic esters of long-chain alkanols are in particular esters of mono-, di- or tricarboxylic acids with long-chain alkanols or polyols, as described in particular in DE-A 38 38 918. The mono-, di- or tricarboxylic acids used may be aliphatic or aromatic acids; suitable ester alcohols or polyols are in particular long-chain representatives having, for example, from 6 to 24 carbon atoms. Typical representatives of the esters are adipates, phthalates, isophthalates, terephthalates and trimellitates of isooctanol, isononanol, isodecanol and isotridecanol, for example di-(n- or isotridecyl) phthalate.

Further suitable carrier oil systems are described, for example, in DE-A 38 26 608, DE-A 41 42 241, DE-A 43 09 074, EP-A 0 452 328 and EP-A 0 548 617, which are explicitly incorporated herein by way of reference.

Examples of particularly suitable synthetic carrier oils are alcohol-started polyethers having from about 5 to 35, for example from about 5 to 30, C₃-C₆-alkylene oxide units, for example selected from propylene oxide, n-butylene oxide and isobutylene oxide units, or mixtures thereof. Nonlimiting examples of suitable starter alcohols are long-chain alkanols or phenols substituted by long-chain alkyl in which the long-chain alkyl radical is in particular a straight-chain or branched C₆-C₁₈-alkyl radical. Preferred examples include tridecanol and nonylphenol.

Further suitable synthetic carrier oils are alkoxylated alkylphenols, as described in DE-A 10 102 913.6.

The inventive compositions may, if appropriate, comprise further coadditives.

Further customary additives are additives which improve the cold properties of the fuel, for example nucleators, flow improvers, paraffin dispersants and mixtures thereof, for example ethylene-vinyl acetate copolymers; corrosion inhibitors, for example based on ammonium salts of organic carboxylic acids, said salts tending to form films, or on heterocyclic aromatics in the case of nonferrous metal corrosion protection; dehazers; antifoams, for example certain siloxane compounds; cetane number improvers (ignition improvers); combustion improvers; antioxidants or stabilizers, for example based on amines such as p-phenylenediamine, dicyclohexylamine or derivatives thereof or on phenols such as 2,4-di-tert-butylphenol or 3,5-di-tert-butyl-4-hydroxyphenylpropionic acid; antistats; metallocenes such as ferrocene; methylcyclopentadienylmanganese tricarbonyl; lubricity improvers, for example certain fatty acids, alkenylsuccinic esters, bis(hydroxyalkyl) fatty amines, hydroxyacetamides or castor oil; and also dyes (markers). Amines are also added if appropriate to lower the pH of the fuel.

When detergent additives, for example those having the polar moieties (a) to (i), are used, they are added to the fuel typically in an amount of from 10 to 5000 ppm by weight, in particular from 50 to 1000 ppm by weight, more preferably from 25 to 500 ppm by weight.

When demulsifiers are used, they are added to the fuel typically in an amount of from 0.1 to 100 ppm by weight, in particular from 0.2 to 10 ppm by weight.

The other components and additives mentioned are, if desired, added in amounts customary for this purpose.

When the inventive additive composition comprises a detergent additive, it is present preferably in an amount of from 1 to 60% by weight, preferably from 1 to 50% by weight, more preferably from 1 to 40% by weight and in particular from 1 to 15% by weight, based on the total weight of the composition.

When the inventive additive composition comprises a demulsifier, it is present preferably in an amount of from 0.01 to 5% by weight, more preferably from 0.01 to 2.5% by weight and in particular from 0.01 to 1% by weight, based on the total weight of the composition.

The inventive compositions may also, if appropriate, also comprise a solvent or diluent.

Suitable solvents and diluents are, for example, aromatic and aliphatic hydrocarbons, for example C₅-C₁₀-alkanes, such as pentane, hexane, heptane, octane, nonane, decane, their constitutional isomers and mixtures; petroleum ether, aromatics such as benzene, toluene, xylenes and Solvent Naphtha; alkanols having from 3 to 8 carbon atoms, for example propanol, isopropanol, n-butanol, sec-butanol, isobutanol and the like, in combination with hydrocarbon solvents; and alkoxyalkanols. Suitable diluents are, for example, also fractions obtained in crude oil processing, such as kerosene, naphtha or brightstock. Diluents used with preference in the case of middle distillates, especially in the case of diesel fuels and heating oils, are naphtha, kerosene, diesel fuels, aromatic hydrocarbons such as Solvent Naphtha heavy, Solvesso® or Shellsol®, and also mixtures of these solvents and diluents.

The individual components may be added to the fuel or to the conventional fuel composition individually or as a concentrate prepared beforehand (additive package; additive composition).

The present invention further also relates to a process for producing at least one fuel composition, wherein a fuel or a fuel composition is admixed
(a) with at least one hydrophobin or derivative thereof and at least one further fuel additive or
(b) with an additive composition as described above.

Hydrophobins or derivatives thereof have good properties in the defoaming of fuels.

The invention is illustrated hereinbelow by examples.

### EXAMPLES

### Example 1

### Preparations for the Cloning of yaad-His₆/yaaE-His₆

A polymerase chain reaction was carried out with the aid of the oligonucleotides Hal570 and Hal571 (Hal 572/Hal 573). The template DNA used was genomic DNA of the bacterium Bacillus subtilis. The resulting PCR fragment comprised the coding sequence of the Bacillus subtilis yaaD/yaaE gene, and an NcoI and BgIII restriction cleavage site respectively at each end. The PCR fragment was purified and cut with the restriction endonucleases NcoI and BgIII. This DNA fragment was used as an insert and cloned into the vector pQE60 from Qiagen, which had been linearized beforehand with the restriction endonucleases NcoI and BgIII. The vectors pQE60YMD#2/pQE60YaaE#5 thus formed may be used to express proteins consisting of YAAD::HIS₆ or YAAE::HIS₆.

Hal570: gcgcgcccatggctcaaacaggtactga
Hal571: gcagatctccagccgcgttcttgcatac
Hal572: ggccatgggattaacaataggtgtactagg
Hal573: gcagatcttacaagtgccttttgcttatattcc

### Example 2

### Cloning of yaad Hydrophobin DewA-His₆

A polymerase chain reaction was carried out with the aid of the oligonucleotides KaM 416 and KaM 417. The template DNA used was genomic DNA of the mold Aspergillus nidulans. The resulting PCR fragment comprised the coding sequence of the hydrophobin gene dewA and an N-terminal factor Xa proteinase cleavage site. The PCR fragment was purified and cut with the restriction endonuclease BamHI. This DNA fragment was used as an insert and cloned into the vector pQE60YAAD#2 which had been linearized beforehand with the restriction endonuclease BgIII.

The vector #508 thus formed can be used to express a fusion protein consisting of YAAD::Xa::dewA::HIS₆.

KaM416:
GCAGCCCATCAGGGATCCCTCAGCCTTGGTACCAGCGC
KaM417:
CCCGTAGCTAGTGGATCCATTGAAGGCCGCATGAAGTTCTCCGTCTCCGC

### Example 3

### Cloning of yaad Hydrophobin RodA-His₆

The plasmid #513 was cloned analogously to plasmid #508 using the oligonucleotides KaM 434 and KaM 435.

KaM434:
GCTAAGCGGATCCATTGAAGGCCGCATGAAGTTCTCCATTGCTGC
KaM435:
CCAATGGGGATCCGAGGATGGAGCCAAGGG

### Example 4

### Cloning of yaad Hydrophobin BASF1-His₆

The plasmid #507 was cloned analogously to plasmid #508 using the oligonucleotides KaM 417 and KaM 418.

The template DNA used was a synthetic DNA sequence (hydrophobin BASF1) (see appendix, SEQ ID NO. 11 and 12).

KaM417:
CCCGTAGCTAGTGGATCCATTGAAGGCCGCATGAAGTTCTCCGTCTCCGC
KaM418:
CTGCCATTCAGGGGATCCCATATGGAGGAGGGAGACAG

### Example 5

### Cloning of yaad Hydrophobin BASF2-His₆

The plasmid #506 was cloned analogously to plasmid #508 using the oligonucleotides KaM 417 and KaM 418.

The template DNA used was a synthetic DNA sequence (hydrophobin BASF2) (see appendix, SEQ ID NO. 13 and 14).

KaM417:
CCCGTAGCTAGTGGATCCATTGAAGGCCGCATGAAGTTCTCCGTCTCCGC
KaM418:
CTGCCATTCAGGGGATCCCATATGGAGGAGGGAGACAG

### Example 6

### Cloning of yaad Hydrophobin SC3-His₆

The plasmid #526 was cloned analogously to plasmid #508 using the oligonucleotides KaM464 and KaM465.

The template DNA used was cDNA from Schyzophyllum commune (see appendix, SEQ ID NO. 9 and 10).

KaM464: CGTTAAGGATCCGAGGATGTTGATGGGGGTGC
KaM465: GCTAACAGATCTATGTTCGCCCGTCTCCCCGTCGT

### Example 7

### Fermentation of the Recombinant E. coli Strain yaad Hydrophobin DewA-His₆

Inoculation of 3 ml of LB liquid medium with a yaad hydrophobin DewA-His₆-expressing E. coli strain in 15 ml Greiner tubes. Inoculation for 8 h at 37° C. on a shaker at 200 rpm. In each case two 1 l Erlenmeyer flasks with baffles and 250 ml of LB medium (+100 μg/ml of ampicillin) are inoculated with 1 ml in each case of the preliminary culture and incubated for 9 h at 37° C. on a shaker at 180 rpm.

Inoculate 13.5 l of LB medium (+100 μg/ml of ampicillin) with 0.5 l of preliminary culture (OD_{600 nm} 1:10, measured against H₂O) in a 20 l fermenter. At an OD_{60 nm} of ˜3.5, addition of 140 ml of 100 mM IPTG. After 3 h, cool fermenter to 10° C. and centrifuge off fermentation broth. Use cell pellet for further purification.

### Example 8

### Purification of the Recombinant Hydrophobin Fusion Protein

(Purification of Hydrophobin Fusion Proteins which have a C-Terminal His6 Tag)

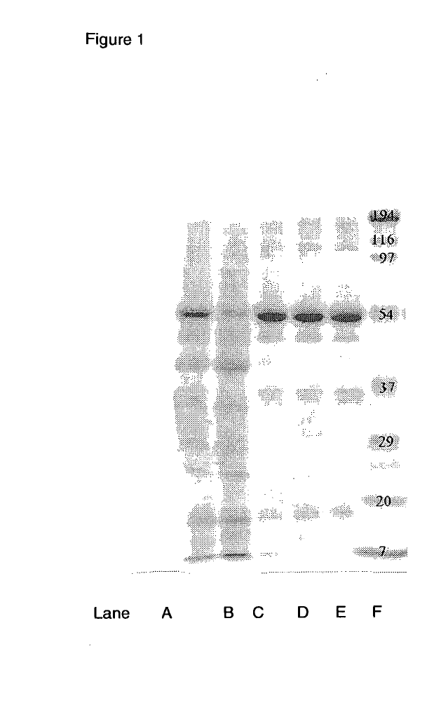

100 g of cell pellet (100-500 mg of hydrophobin) are made up to total volume 200 ml with 50 mM sodium phosphate buffer, pH 7.5, and resuspended. The suspension is treated with an Ultraturrax type T25 (Janke and Kunkel; IKA-Labortechnik) for 10 minutes and subsequently incubated with 500 units of Benzonase (Merck, Darmstadt; order No. 1.01697.0001) at room temperature for 1 hour to degrade the nucleic acids. Before the cell disruption, filtration is effected with a glass cartridge (P1). For cell disruption and for the scission of the remaining genomic DNA, two homogenizer cycles are carried out 1500 bar (Microfluidizer M-110EH; Microfluidics Corp.). The homogenate is centrifuged (Sorvall RC-5B, GSA rotor, 250 ml centrifuge cup, 60 minutes, 4° C., 12 000 rpm, 23 000 g), the supernatant was placed on ice and the pellet was resuspended in 100 ml of sodium phosphate buffer, pH 7.5. Centrifugation and resuspension are repeated three times, the sodium phosphate buffer comprising 1% SDS at the third repetition. After the resuspension, the mixture is stirred for 1 hour and a final centrifugation is carried out (Sorvall RC-5B, GSA rotor, 250 ml centrifuge cup, 60 minutes, 4° C., 12 000 rpm, 23 000 g). According to SDS-PAGE analysis, the hydrophobin is present in the supernatant after the final centrifugation (FIG. 1). The experiments show that the hydrophobin is probably present in the form of inclusion bodies in the corresponding E. coli cells. 50 ml of the hydrophobin-comprising supernatant are applied to a 50 ml nickel Sepharose High Performance 17-5268-02 column (Amersham) which has been equilibrated with 50 mM Tris-Cl pH 8.0 buffer. The column is washed with 50 mM Tris-Cl pH 8.0 buffer and the hydrophobin is subsequently eluted with 50 mM Tris-Cl pH 8.0 buffer which comprises 200 mM imidazole. To remove the imidazole, the solution is dialyzed against 50 mM Tris-Cl pH 8.0 buffer.

FIG. 1 shows the purification of the hydrophobin prepared:

Lane A: Application to nickel Sepharose column (1:10 dilution)

Lane B: Flow-through=washing step eluate

Lanes C-E: OD 280 Maxima of the elution fractions (WP1, WP2, WP3)

Lane F shows the applied marker.

The hydrophobin of FIG. 1 has a molecular weight of approx. 53 kD. Some of the smaller bands represent degradation products of the hydrophobin.

### Example 9

### Performance Testing; Characterization of the Hydrophobin by Change in Contact Angle of a Water Drop on Glass

### Substrate:

Glass (window glass, Süddeutsche Glas, Mannheim):
hydrophobin concentration: 100 μg/ml
incubation of glass plates overnight (temperature 80° C.) in 50 mM sodium acetate pH 4+0.1% Tween 20
then wash coating in distilled water
then incubation 10 min/80° C./1% SDS solution in dist. water
Wash in dist. water

The samples are dried under air and the contact angle (in degrees) of a drop of 5 μl of water is determined.

The contact angle was measured on a Dataphysics Contact Angle System OCA 15+, Software SCA 20.2.0. (November 2002). The measurement was effected in accordance with the manufacturer's instructions.

Untreated glass gave a contact angle of 30±5°; a coating with the functional hydrophobin according to example 8 (yaad-dewA-his₆) gave contact angles of 75±5°.

### Example 10

### Use of a Hydrophobin Concentrate (Yaad-dewA-His₆) as a Defoamer

The improvement in defoaming was carried out by means of a hand-shake foaming test as follows:
100 ml of fuel or additized fuel were introduced into a 250 ml screwtop glass bottle which was sealed tightly;
the sample was shaken for 2 min,
the sample was then put down immediately and the volume of the foam (ml) the decomposition time of the foam (sec) were determined.

For the experiment, a hydrophobin concentrate (Yaad-dewA-His₆, as a solution in NaH₂PO₄ buffer (50 mmol/L, pH 7.5)) was used. The starting sample had a concentration of 6.1 mg/ml of hydrophobin. 2 mL of the starting sample were made up to 100 ml (Hyd. sol. 1), and 3 mL of the resulting solution were added to 97 mL of fuel (EN 590 fuel). The results of the experiment are reproduced in the table which follows.

Foam according to Repsol

| | Dosage | DK | Volume | Break time |
|---|---|---|---|---|
| Unadditized | | 976 | 10 | 18 |
| Hyd. sol 2 | 3 mL | 976 | 0 | 0 |

The amount of foam and the foam decomposition time were lower in the case of additization with the hydrophobin concentrate than when the diesel fuel did not comprise any hydrophobin.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A process for defoaming fuel comprising adding at least one hydrophobin or derivative thereof to the fuel.

13. The process of claim 12, wherein the fuel is a diesel fuel.

14. The process of claim 12, wherein the at least one hydrophobin or derivative thereof comprises 0.1 to 100 ppm of the fuel.

15. The process of claim 12, wherein the at least one hydrophobin or derivative thereof comprises 0.15 to 50 ppm of the fuel.

16. The process of claim 12, wherein the at least one hydrophobin or derivative thereof comprises from 0.2 to 30 ppm of the fuel.

17. The process of claim 12, wherein the at least one hydrophobin or derivative thereof comprises from 0.3 to 10 ppm of the fuel.

18. An additive composition comprising at least one hydrophobin or derivative thereof and at least one further fuel additive.

19. The additive composition of claim 18, wherein the at least one further fuel additives comprises a demulsifier.

20. The additive composition of claim 18, wherein the at least one further fuel additive comprises a detergent.

21. The additive composition of claim 20, wherein the at least one further fuel additive further comprises a demulsifier.

22. A fuel composition comprising a fuel as a main constituent, at least one hydrophobin or derivative thereof, and at least one further fuel additive.

23. The fuel composition of claim 22, wherein the fuel is diesel fuel.

24. The fuel composition of claim 22, wherein the at least one further fuel additive is selected from the group consisting of a detergents, demulsifiers, additives that improve the cold properties of the fuel composition, corrosion inhibitors, dehazers; antifoams, cetane number improvers, combustion improvers, antioxidants, stabilizers, antistats, metallocenes, methylcyclopentadienylmanganese tricarbonyl, lubricity improvers, dyes, and amines.

25. A process for producing a fuel composition comprising mixing a fuel or a fuel composition with a hydrophobin or derivative thereof and at least one fuel additive.

26. The process of claim 25, wherein the at least one fuel additive is selected from the group consisting of a detergent and a demulsifier.
